(19) United States
(12) Plant Patent Application Publication
(10) Pub. No.: US 2013/0074225 P1
(43) Pub. Date: Mar. 21, 2013

(54) Apple Tree Named 'PremA96'

(50) ;

(71) Applicant: Allan White, Havelock North (NZ)

(72) Inventor: Allan White, Havelock North (NZ)

(21) Appl. No.: 13/441,751

(22) Filed: Apr. 6, 2012

Related U.S. Application Data
(60) Provisional application No. 61/517,139, filed on Apr. 13, 2011.

(51) Int. Cl. A01H 5/00

(52) U.S. Cl. PLT/161

(57) ABSTRACT

A new and distinct apple tree named ‘PremA96’ is disclosed. The new apple is notable for its attractive appearance, superb texture, improved flavor and long shelf life.

### LATIN NAME OF THE GENUS AND SPECIES OF THE PLANT CLAIMED

Mallus domestica Mill

### VARIETY DENOMINATION

‘PREMA96’

### BRIEF DESCRIPTION OF THE PHOTOGRAPHS

FIG. 1 shows the fruit of a ‘PremA96’ apple tree;

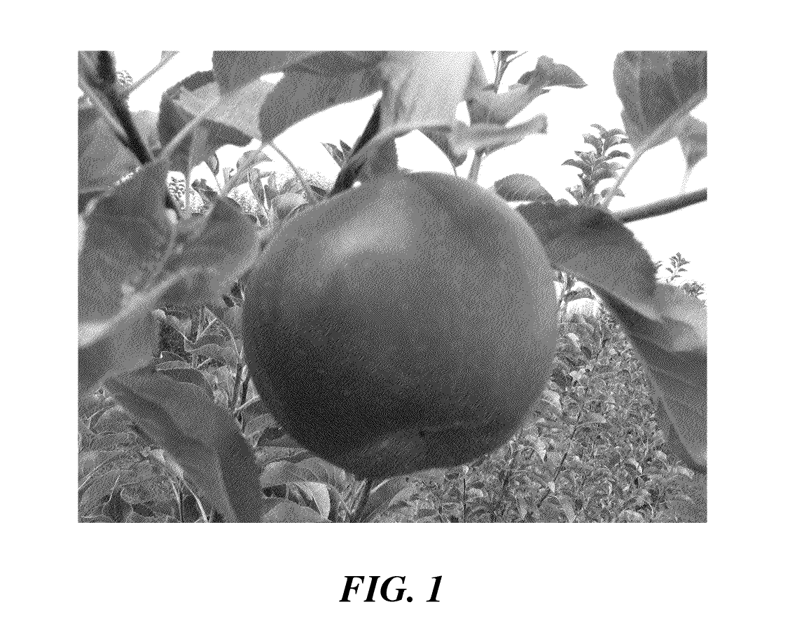

FIG. 2 shows a ‘PremA96’ apple tree;

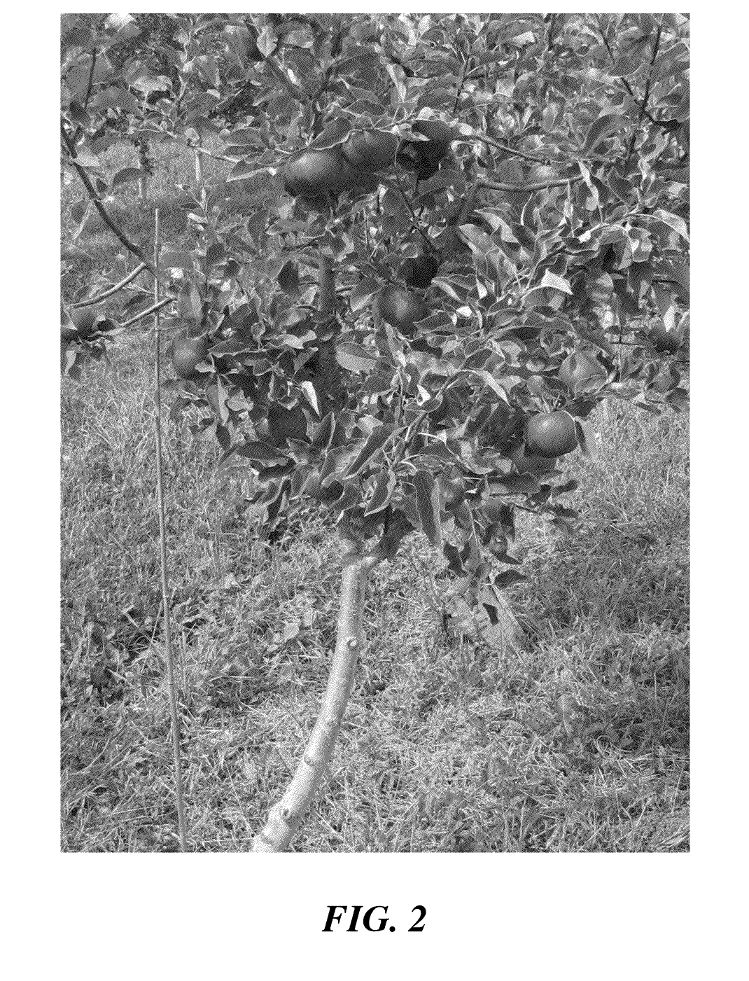

### BRIEF DESCRIPTION OF THE VARIETY

The new variety of apple tree ‘PremA96’ was created in the course of a planned breeding program carried out at The New Zealand Institute for Plant and Food Research, Hawkes Bay, New Zealand. ‘PremA96’ originated as a result of a controlled cross of ‘Royal Gala’ (U.S. Plant Pat. 4,121, patent expired) and ‘GS 2184’ (pollen parent, not patented). ‘PremA96’ was selected in 1996 as a single plant from among a population of seedlings derived from the parents, and was selected for its attractive red color and unique appearance, superb texture and flavor, and long shelf life. Asexual propagation by budding was first carried out in Nelson, New Zealand. The variety has since been observed over a number of asexually propagated generations, and has been found to remain true to type.

### DETAILED BOTANICAL DESCRIPTION OF THE VARIETY

The following detailed botanical description is based on observations made during the 2011 growing season at Parker, Wash. All colors are described according to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Age of observed variety — 4 years; Habit — Spreading; Size — Height 219 cm, spread 143 cm; Trunk — Diameter at 30 cm above graft union 4 cm; Bark texture — smooth; Bark overcolor — 198D, Bark undercolor — 166D; Lenticels diameter 0.4 cm, Lenticels color 155D.
Branch: Size — Length 30.7 cm, Diameter 0.9 cm at 1 m above graft union; Crotch angle relative to trunk — 50°; Branch color — 165A; Lenticel size — 0.1 cm diameter , Lenticel color 155D, Lenticel density — 13 per square inch;
Dormant one-year-old shoot: Size — length 43 cm, diameter 0.3 cm; Color — 165A; Internode length — 2.1 cm; Lenticels — quantity 24 per square inch, color 155D, diameter 0.1 cm
Winter hardiness: Hardy for Washington State; Chilling Requirement — Medium
Leaf: Blade margin — serrated; Size — length 8.2 cm, width 4.9 cm; Color — upper surface 143C; Color of lower surface — 143D; Attitude in relation to shoot — 50°.
Petiole: Length 1.6 cm; Diameter 0.2 cm; Color 63A.
Fruit: Quantity per cluster — 3-5; Size — diameter 7 cm, length 6.6 cm; General shape in profile globose; Aperture of eye 2 cm; Depth of eye basin 1 cm; Stalk — diameter 0.3 cm, length 3 cm; Depth of stalk cavity 0.5 cm; Width of stalk cavity 1 cm; Size of lenticels 0.1 cm; Ground color of skin Yellow 3A; Over color of skin Orange N25A to Red 47A; Amount of over color 90%; Intensity of over color — medium; Pattern of over color — blush; Flesh texture is fine and very juicy; Flesh color 11C; Quantity of seeds 9; Seed shape — Length 0.9 cm, Diameter 0.5 cm; Seed color 16A.
Harvest: 1^{st} to 2^{nd} week of September in Parker, Wash.
Use: Fresh Market.

1. We claim a new and distinct apple tree substantially as shown and described herein.
